(19) United States
(12) Patent Application Publication
(10) Pub. No.: US 2011/0307040 A1
(43) Pub. Date: Dec. 15, 2011

(54) THERAPEUTIC HEAT-TRANSFER PACK

(76) Inventor: Samuel Brian Peterson, Singapore (SG)

(21) Appl. No.: 12/814,736

(22) Filed: Jun. 14, 2010

(51) Int. Cl. A61F 7/03; A61F 7/02

(52) U.S. Cl. 607/108; 607/114; 607/112

(57) ABSTRACT

A manufacture for placement against a target site includes a bag containing a bag filling. The bag has an inner surface and an outer surface. The bag filling is in contact with the inner surface. A hydrogel layer adheres to a first portion of the outer surface of the bag.

### FIELD OF DISCLOSURE

This disclosure relates to physical therapy devices, and in particular, to heating and cooling selected areas of the body.

### BACKGROUND

To relieve pain and promote healing, it is known to either heat or cool a therapy site on the body. This is typically achieved by applying a hot pack or a cold pack (collectively referred to herein as a heat-transfer pack) to the therapy site.

A difficulty that arises is that of fixing the heat-transfer pack to the therapy site. One known way to do so is to strap the pack to the therapy site using adhesive tape. However, this adhesive tape can be painful to remove and difficult to position on some parts of the body. As an alternative to strapping the pack onto the therapy site with adhesive tape, it is also known to place the pack in a pouch, and to provide an adhesive on the pouch. However, while this reduces the adhesive contact area to that of the pouch only, the pain of removal, particularly on the more hirsute portions of the body, remains. In addition, the intervening pouch interferes with heat transfer due to added material and potential air gaps between heat transfer material and the therapy site. Such a pouch also adds to the complexity of use.

### SUMMARY

The invention is based on the recognition that hydrogel is adhesive enough to hold a heat-transfer pack to a selected area of the body but not so adhesive as to cause significant pain during removal of the heat-transfer pack.

In one aspect, the invention features a manufacture for placement against a target site. Such a manufacture includes a bag containing a bag filling. The bag has an inner surface and an outer surface. The bag filling is in contact with the inner surface. A hydrogel layer adheres to a first portion of the outer surface of the bag.

In some embodiments, a second portion of the bag is made of an viscoelastic material. Among these embodiments are those in which the viscoelastic material includes a viscoelastic polymer.

Alternative embodiments of the invention include those in which the bag has been preformed to conform to a body part. One such embodiment is that in which the bag is preformed to conform to a human shoulder.

In other embodiments, the manufacture also includes a removable protective film to cover a free surface of the hydrogel layer. Exemplary films include impermeable films.

Embodiments of the invention also include those in which the bag has a spatially varying heat transfer coefficient. Examples of these embodiments include those in which the inner surface and the outer surface are separated by a distance that varies as a function of position on the bag, those in which the bag includes a first bag material and a second bag material, with a heat transfer coefficient of the first bag material being greater than a heat transfer coefficient of the second bag material, and those in which the bag is made of a bag material and includes a heat mask disposed over a selected portion of the bag, whereby a heat transfer coefficient of the heat mask and the bag material is less than a heat transfer coefficient of the bag material.

Other embodiments include those in which the bag has a spatially constant heat transfer coefficient.

In an alternative embodiment, the bag filling includes reagents that, when mixed, cause a exothermic or endothermic chemical reaction, as well as those in which the bag filling includes a water gel.

The invention includes within its scope hydrogels of varying adhesivity between hydrogel and skin. Examples include embodiments in which the hydrogel has adhesivity between hydrogel and skin is approximately 400 GPIW, those in which the hydrogel has an adhesivity between hydrogel and skin that is between about 25 GPIW and 1000 GPIW, those in which the adhesivity between hydrogel and skin is greater than about 25 grams GPIW, and those in which the hydrogel is selected such that the adhesivity between hydrogel and skin is less than about 1000 GPIW.

Other embodiments include those in which the hydrogel is selected to have an adhesivity sufficient to maintain the bag's position on skin having a normal vector orthogonal to a gravity vector for a therapeutically significant period.

In another aspect, the invention features a kit including a first manufacture and a second manufacture according, each of which is an embodiment of the invention described above, and in which each the manufactures have different heat transfer coefficients.

These and other features of the invention will be apparent from the following detailed description and the accompanying figures, in which:

### BRIEF DESCRIPTION OF THE FIGURES

FIG. 1 shows a therapeutic pack having a bag that adheres to a hydrogel layer;

FIG. 2 shows a therapeutic pack shaped to conform to a shoulder;

FIG. 3 shows a therapeutic pack shaped for application to the temples;

FIG. 4 shows a therapeutic pack shaped for application around the eyes;

FIG. 5 shows a therapeutic pack in which the bag is made of different materials for applying heat in an annulus; and

FIG. 6 shows a cross-section of a therapeutic pack in which the bag has a locally varying thickness.

### DETAILED DESCRIPTION

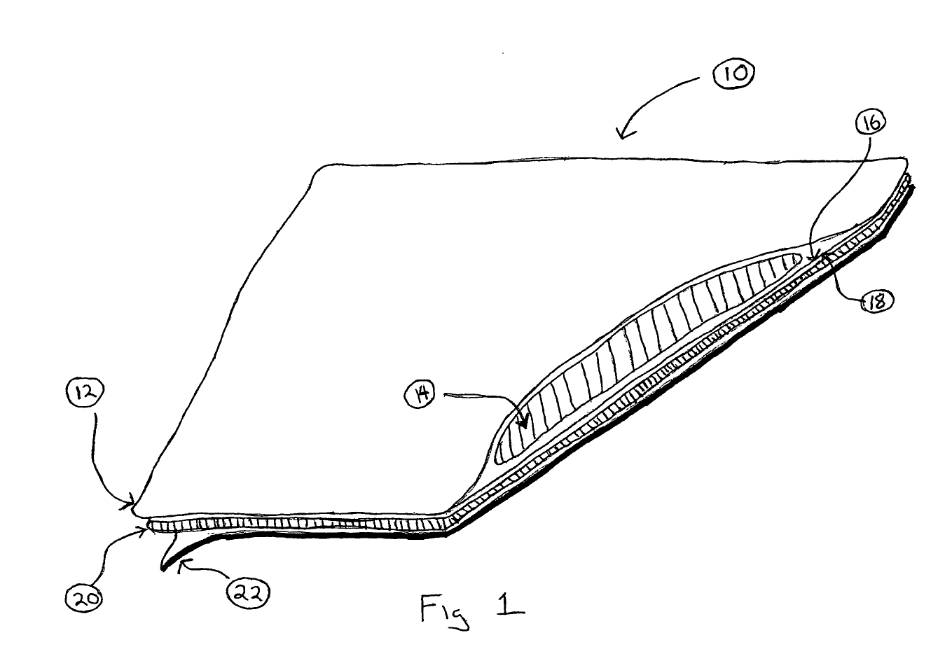

Referring to FIG. 1, a heat-transfer pack 10 features a flexible and impermeable bag 12 that defines a volume filled with a filling 14. The filling 14 is typically a gel or fluid that functions as a heat source or a heat sink Examples of fillings include reagents that, when combined, cause a chemical reaction. Such a reaction could be an exothermic or endothermic reaction depending on whether the heat-transfer pack 10 is intended to warm or cool a therapy site. Alternatively, the filling 14 can be a passive filling having a high heat capacity. Such a filling 14 would then be heated, for example in a microwave oven, or cooled, for example in a refrigerator or freezer, prior to use on the therapy site.

A typical passive filling is a water gel, which typically comprises superabsorbent polymer, water, and salt. Because of its high specific heat, the acts as a reusable cooling or heating agent. The superabsorbent polymer retains the water in a gel form (i.e. a hydrogel) to enable it to partially retain its shape within the bag, as well as to minimize spillage in case the bag develops a puncture. Superabsorbent polymer is commonly made from the polymerization of acrylic acid blended with sodium hydroxide in the presence of an initiator to form a poly-acrylic acid sodium salt. Other fillings are also used to make a superabsorbent polymer, such as polyacrylamide copolymer, ethylene maleic anhydride copolymer, cross-linked carboxy-methyl-cellulose, polyvinyl alcohol copolymers, cross-linked polyethylene oxide, and starch grafted copolymer of polyacrylonitrile. The salt is keeps the gel from crystallizing (solidifying) at household freezer temperatures (0-5 degrees Fahrenheit). Flexibility of the gel is a desirable property useful in moulding the bag to body contours.

As used herein, “therapy site” is used broadly to include sites affected by some injury. For example, in the case of a strained ligament, the therapy site would include the skin near the strained ligament. However, the purpose of the application need not involve any injury. For instance, it is known to apply heating or cooling purely for relaxation or regulating body temperature, e.g., fever control or hypothermia therapy.

The bag 12 has an inner surface 16 and an outer surface 18. A suitable material for the bag 12 is polypropylene or polyethylene, nylon or some combination thereof.

However, any flexible material having a desired heat transfer coefficient can be used Preferably, the bag is a puncture resistant bag that can be heat sealed on industry standard equipment in a way that results in a heat seal strong enough to endure many hot-cold cycles ranging from 0 to 250 degrees Fahrenheit.

To facilitate heat transfer, as well as to simplify construction, it is useful to minimize the number of layers that would impede heat transfer between the filling 14 and the therapy site. Thus, the inner surface 16 of the bag 12 is in direct contact with the filling 14, and a first portion of the bag's outer surface 18 adheres to a hydrogel layer 20. This hydrogel layer 20 adheres to the skin at the therapy site. As a result, there are only two intervening layers between the filling 14 and the therapy site: the bag 12 and the hydrogel layer 20. This provides a uniform and controlled heat transfer surface, and thus avoids introducing air pockets, that would disrupt the heat transfer between the filling and the therapy site. Such air pockets may arise, for example, if a bag is inserted into a separate pouch which is then applied to the therapy site.

The hydrogel layer 20 is sufficiently adhesive to hold the first portion of the bag 12 to the skin However, its adhesiveness is low enough to avoid discomfort as the hydrogel layer is peeled off the skin In addition, because of its high thermal conductivity, hydrogel is particularly well suited for being an intermediate layer between the bag 12 and the therapy site.

The hydrogel adhesive is formulated and cross-linked to provide weak adhesion to the skin By varying the extent of cross linking within the hydrogel, one can control the extent to which it is adhesive, hereafter referred to as its “adhesivity”

Preferably, the hydrogel has an adhesivity that is sufficient to hold the bag to the skin when a vector normal to the skin is perpendicular to the gravity vector for a therapeutically significant amount of time, for example at least about twenty minutes. Adhesivities of hydrogel are often measured by the amount of force required to peel off a one inch strip of hydrogel from a smooth steel surface. Suitable ranges are adhesivities greater than about 25 GPIW (grams per inch of width), and greater than about 75 GPIW but less than about 1700 GPIW. Hydrogels having adhesivities on the order of 25 GPIW to 300 GPIW have been found to offer a good compromise between adhesive strength and relatively painless removal. An adhesivity of about 400 GPIW has been found to be suitable for use. Additional ranges of adhesivity that can be used are adhesivities greater than about 25 GPIW, adhesivities less than about 1000 GPIW, and adhesivities in the range between about 25 GPIW and about 1000 GPIW.

Suitable hydrogels are those sold by Katecho, Inc. Among these hydrogels is one sold under the tradenames KM30D, which has an adhesivity of 397 GPIW.

Unlike conventional adhesives, hydrogel can be used repeatedly with little or no diminution in its ability to adhere to the skin Thus, the article can repeatedly be removed from the therapy site and returned to the therapy site. To protect the hydrogel layer 20 from dirt and dust during storage, it is useful to provide a removable film 22. Such a film 22 is preferably a non-porous film that slows or prevents water from escaping the hydrogel layer 20.

In some embodiments, a second portion of the bag 12, which does not adhere to the hydrogel layer 20, is made of an elastomer or another material having elastic properties. Such embodiments are useful when the therapy site has a low radius of curvature. In such cases, the bag 12 is bent to conform to a surface having a low radius of curvature. Under such conditions, the second portion may exert enough force on the first portion to cause the heat transfer pack to partially peel from the therapy site. A second portion with some elasticity will be able to stretch instead of exerting such a force. For convenience in manufacturing, the entire bag 12, including the first and second portions, can be made of an elastomer.

In some cases, the therapy site is one with an irregular shape. In such cases, a conventional pack is inconvenient because it cannot easily be molded to conform to the desired shape. For example, if the therapy site is the shoulder, it may be difficult to bend the heat-transfer pack 10 to conform to the shape of the shoulder.

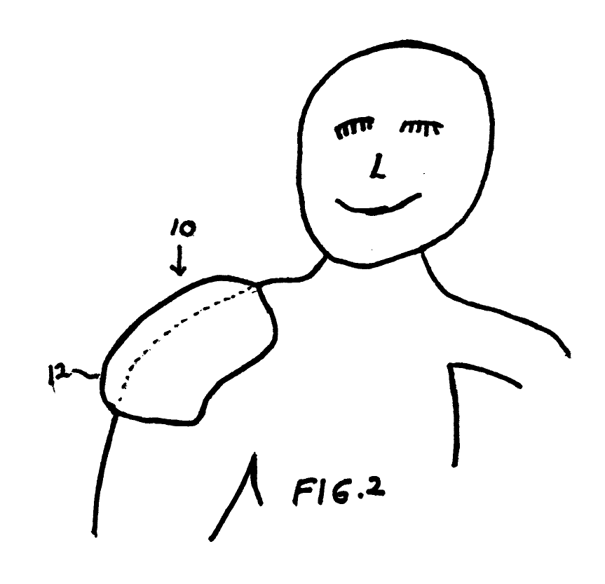

In an alternative embodiment, shown in FIG. 2, the bag 12 and the accompanying hydrogel layer 20 on its first portion are pre-molded to conform to the shape of a therapy site. For example, the bag 12 can be molded to conform to the shape of a shoulder, elbow, crotch, or a knee. Or the bag 12 can be molded to conform to the shape of a neck or collar.

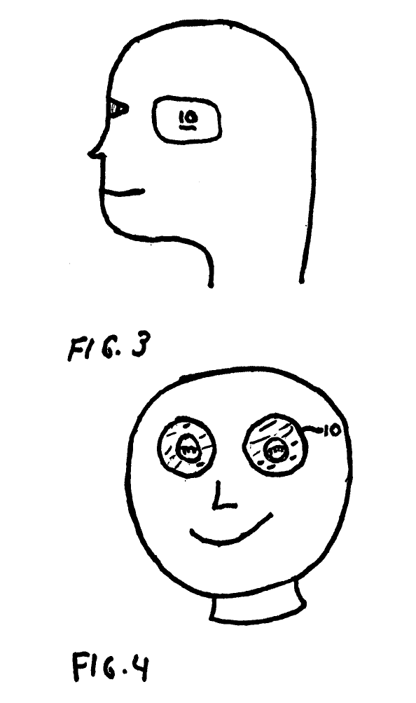

A heat-transfer pack 10 that adheres using a hydrogel layer 20 can also be made to adhere to portions of the body that would otherwise be inconvenient using conventional large rectangular packs. For example, a small heat-transfer pack 10 can adhere to the temple, as shown in FIG. 3, to apply heat or cold to relieve headaches. Or a heat-transfer pack 10 can be made in the shape of a donut to be placed over and apply heat around an eye, as shown in FIG. 4.

In some applications, it is desirable to apply greater or lesser amounts of heat or cold to certain localized regions within a therapy site. To achieve this, certain embodiments feature spatially varying heat transfer coefficients.

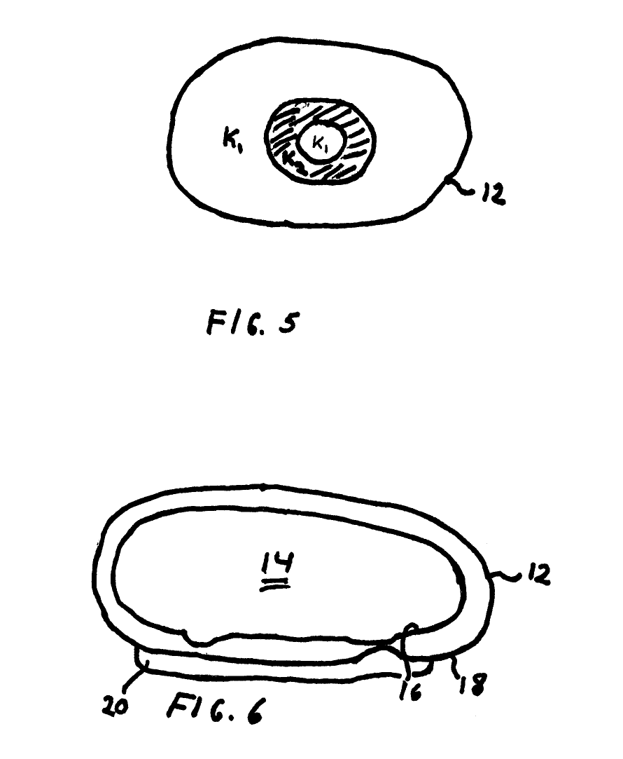

Spatially varying heat transfer coefficients can be achieved in a variety of ways. One way, shown in FIG. 5, is to manufacture an inhomogeneous heat-transfer pack 10, in which materials with low heat transfer coefficients are used for those regions in which limited heat transfer is sought, and in which materials with higher heat transfer coefficients are used in those regions in which greater heat transfer is sought. A similar result can be achieved, as shown in FIG. 6, by using one material and spatially varying its thickness depending on whether greater or less heat transfer is sought. As shown in FIG. 6, this results in locally thin areas, where greater heat transfer is sought, and locally thick areas, were less heat transfer is sought. Another way to achieve this result is to insert an insulating mask between the bag and the hydrogel layer. As a result, the heat transfer coefficient for the combination of the mask material and the bag material would be less than the heat transfer coefficient of the bag material by itself.

Heat transfer packs along the lines of the foregoing can be sold in kits that include two or more bags, each one having a different heat transfer coefficient. Using such a kit, one can select the appropriate bag for a particular injury or therapy. For example, if circulation is poor, a bag with an excessive heat transfer coefficient may result in frostbite or a burn. In such cases it would be useful to select a bag having a more appropriate heat transfer coefficient from the kit.

Heat transfer packs along the lines of the foregoing can also be used for non-therapeutic applications. For example, one can use such packs to warm up or cool down a target site. As one example one can use such a pack to absorb heat generated by a chemical reaction, or to cool or heat foods, or to cool industrial equipment.

Having described the invention, and a preferred embodiment thereof, what I claim as new, and secured by letters patent is:

1. A manufacture for placement against a target site, said manufacture comprising:
a bag containing a bag filling, said bag having an inner surface and an outer surface, said bag filling being in contact with said inner surface; and
a hydrogel layer adhering to a first portion of said outer surface of said bag.

2. The manufacture of claim 1, wherein a second portion of said bag is made of an viscoelastic material.

3. The manufacture of claim 2, wherein said viscoelastic material comprises a viscoelastic polymer.

4. The manufacture of claim 1, where said bag is preformed to conform to the shape of a body part.

5. The manufacture of claim 4, wherein said bag is preformed to conform to a human shoulder.

6. The manufacture of claim 1, further comprising a removable protective film to cover a free surface of said hydrogel layer.

7. The manufacture of claim 6, wherein said protective film is impermeable.

8. The manufacture of claim 1, wherein said bag has a spatially varying heat transfer coefficient.

9. The manufacture of claim 8, wherein said inner surface and said outer surface are separated by a distance that varies as a function of position on said bag.

10. The manufacture of claim 8, wherein said bag comprises a first bag material and a second bag material, and wherein a heat transfer coefficient of said first bag material is greater than a heat transfer coefficient of said second bag material.

11. The manufacture of claim 8, wherein said bag is made of a bag material and comprises a heat mask disposed over a selected portion of said bag, whereby a heat transfer coefficient of said heat mask and said bag material is less than a heat transfer coefficient of said bag material.

12. The manufacture of claim 1, wherein said bag has a spatially constant heat transfer coefficient.

13. The manufacture of claim 1, wherein said bag filling comprises reagents that, when mixed, cause a exothermic or endothermic chemical reaction.

14. The manufacture of claim 1, wherein said bag filling comprises a water gel.

15. The manufacture of claim 1, wherein the hydrogel is selected such that the adhesivity between hydrogel and skin is approximately 400 GPIW.

16. The manufacture of claim 1, wherein the hydrogel is selected such that the adhesivity between hydrogel and skin is between about 25 GPIW and 1000 GPIW.

17. The manufacture of claim 1, wherein the hydrogel is selected such that the adhesivity between hydrogel and skin is greater than about 25 grams GPIW.

18. The manufacture of claim 1, wherein the hydrogel is selected such that the adhesivity between hydrogel and skin is less than about 1000 GPIW.

19. The manufacture of claim 1, wherein said hydrogel is selected to have an adhesivity sufficient to maintain said bag's position on skin having a normal vector orthogonal to a gravity vector for a therapeutically significant period.

20. A kit comprising a first manufacture according to claim 1 and a second manufacture according to claim 1, where said first manufacture comprises a bag having a first heat transfer coefficient and said second manufacture comprises a bag having a second heat transfer coefficient that is greater than said first heat transfer coefficient.
